(19) United States
(12) Patent Application Publication
(10) Pub. No.: US 2023/0218363 A1
(43) Pub. Date: Jul. 13, 2023

(54) SYSTEM FOR MONITORING A SURGICAL LUMINAIRE ASSEMBLY

(71) Applicant: KARL LEIBINGER MEDIZINTECHNIK GMBH & CO. KG, Muehlheim (DE)

(72) Inventors: Joachim STROELIN, Rietheim-Weilheim (DE); Harald SCHULZ, Tuttlingen (DE)

(21) Appl. No.: 18/000,268

(22) PCT Filed: May 27, 2021
(86) PCT No.: PCT/EP2021/064205

§ 371 Date: Nov. 29, 2022

(30) Foreign Application Priority Data
May 29, 2020 (DE) 10 2020 114 425.3

(51) Int. Cl. A61B 90/35; F21V 23/04; F21V 25/00

(52) U.S. Cl. CPC A61B 90/35; F21V 23/0464; F21V 25/00; A61B 2090/309

(57) ABSTRACT

The present invention comprises a system for monitoring a surgical luminaire assembly comprising at least one surgical luminaire, with a monitoring unit. The monitoring unit determines an energy input of the at least one surgical luminaire into the surgical field.

The present invention relates to the operation of a surgical luminaire assembly comprising at least one surgical luminaire or at least two surgical luminaires.

Surgical luminaires of new design have a very high performance potential. The maximum illuminance as well as the maximum irradiance are capped by the surgical luminaire standard 60601-2-41. The standard stipulates a maximum irradiance of 1000 W/m² per surgical luminaire unit. However, this limit is not sufficient to reliably prevent heating of the surgical field and the associated accelerated tissue drying.

It is therefore an object of the present invention to provide a system which enables safer operation of the surgical luminaire assembly.

This object is achieved by systems for monitoring a surgical luminaire assembly according to claims 1 and 7. Preferred embodiments of the present invention are the subject-matter of the dependent claims.

In a first aspect, the present invention comprises a system for monitoring a surgical luminaire assembly comprising at least one surgical luminaire, with a monitoring unit. The monitoring unit determines an energy input of the at least one surgical luminaire into the surgical field. In particular, the energy input is the total power introduced into the surgical field by the at least one surgical luminaire.

The present invention is also based on the knowledge, that the irradiance limited by the standard is only measured in the center of the light field and therefore does not represent an indication of the total irradiance in the entire light field or the total power and thus the energy input into the surgical field, and that this energy input represents an important parameter for the heating of the surgical field and tissue drying. By determining the energy input by the monitoring unit according to the invention, it is now possible to detect excessive energy input into the surgical field and avoid the associated problems.

In one possible embodiment of the present invention, the surgical luminaire has an illuminance of at least 40 kLux and/or a light field diameter of at least 100 mm.

Preferably, the surgical luminaire has an illuminance of at least 80 kLux and/or a light field diameter of at least 200 mm, in particular an illuminance of at least 120 kLux and/or a light field diameter of at least 300 mm.

If the illuminance and/or the light field diameter of the surgical luminaire is adjustable, the above statements relate to the fact that the illuminance and/or the light field diameter can be set at least to these values, i.e. according to the above embodiment, it can be set to an illuminance of at least 40 kLux, preferably at least 80 kLux and further preferably at least 12 kLux and/or a light field diameter of at least 100 mm, preferably at least 200 mm and further preferably at least 300 mm.

In one possible embodiment of the present invention, the surgical luminaire can be arranged above an operating table to illuminate a surgical field.

In one possible embodiment of the present invention, the monitoring unit determines the energy input as a function of a light field diameter and an illuminance of the at least one surgical luminaire. Preferably, the monitoring unit evaluates parameters of a control apparatus of the at least one surgical luminaire for this purpose.

In one possible embodiment of the present invention, the surgical luminaire comprises a plurality of light sources, in particular LEDs, wherein the monitoring unit determines the light field diameter and/or the illuminance on the basis of the actuation of the light sources.

In one possible embodiment of the present invention, the light field diameter and/or the illuminance of the at least one surgical luminaire is adjustable.

The light field diameter and/or the illuminance can be variable continuously and/or in steps.

In one possible embodiment of the present invention, the light field diameter of the at least one surgical luminaire is variable by at least 25 mm, preferably by at least 50 mm, more preferably by at least 100 mm. In other words, in this embodiment, the difference between the smallest settable light field diameter and the largest settable light field diameter is at least 25 mm, preferably at least 50 mm, more preferably by at least 100 mm.

In one possible embodiment of the present invention, the light field diameter of the at least one surgical luminaire is adjustable at least between a light field diameter range of 200 mm and 300 mm, preferably between 140 mm and 350 mm. In other words, in this embodiment, the light field diameter can be set both to a value of less than or equal to 200 mm, preferably less than or equal to 140 mm, and to a value of 300 mm or more, preferably 350 mm or more.

In one possible embodiment of the present invention, the illuminance of the at least one surgical luminaire is variable by at least 20 kLux, preferably by at least 40 kLux. In other words, in this embodiment, the difference between the smallest settable illuminance and the greatest settable illuminance is at least20 kLux, preferably at least 40 kLux, more preferably around at least 60 kLux.

In one possible embodiment of the present invention, the illuminance of the at least one surgical luminaire can be set at least in an illuminance range between 80 kLux and 120 kLux and preferably between 40 kLux and 160 kLux. In other words, in this embodiment, the illuminance can be set both to a value of less than or equal to 80 kLux, preferably less than or equal to 40 kLux, and to a value of 120 kLux or more, preferably 160 kLux or more.

Preferably, the light field diameter and/or the illuminance can be varied continuously and/or in several steps over the specified ranges.

In one possible embodiment of the present invention, it is provided that the monitoring unit redetermines the energy input in the event of a change in the light field diameter and/or the illuminance.

In particular, the at least one surgical luminaire can have a control apparatus via which the light field diameter and/or the illuminance of the at least one surgical luminaire can be adjusted, in particular via input elements of the control unit, wherein the monitoring unit evaluates the new parameters of the control apparatus and redetermines the energy input when the light field diameter and/or the illuminance change.

In one possible embodiment of the present invention, the monitoring unit determines an expected temperature rise of the surgical field on the basis of the energy input.

In one possible embodiment of the present invention, the monitoring unit determines the expected temperature rise of the surgical field on the basis of the energy input and a light field diameter of the at least one surgical luminaire.

In one possible embodiment of the present invention, the monitoring unit determines the expected temperature rise of the surgical field as a function of a parameter of the surgical field, which the monitoring unit determines via a sensor, in particular an optical sensor.

In particular, a color of the surgical field can be detected here by a sensor, for example a camera, and taken into account when determining the expected temperature rise. In this way, the higher light absorption of darker tissue can be taken into account. The sensor can, for example, be arranged on the at least one surgical luminaire, and in particular can be arranged on a handle of the surgical luminaire and/or can be directed towards the center of the light field of the surgical luminaire.

In a possible embodiment of the present invention, it is provided that the monitoring unit displays the energy input into the surgical field and/or a value determined on the basis of the energy input into the surgical field and/or monitors for exceedance of a limit value.

In particular, it can be provided that the temperature rise is displayed by the monitoring unit and/or monitored for exceedance of a limit value.

In one possible embodiment of the present invention, it is provided that the monitoring unit emits an acoustic and/or optical warning and/or automatically limits or reduces the illuminance when a limit value is exceeded.

In particular, this can be a limit value for the energy input and/or an expected temperature rise of the surgical field.

In one possible embodiment of the present invention, the system comprises an input unit via which the limit value can be adjusted by an operator.

In one possible embodiment of the present invention, a monitoring and/or control function of the monitoring unit can be switched on and off.

In one possible embodiment of the present invention, the type of display and/or response to the limit value being exceeded is selectable.

In a second independent aspect, the present invention comprises a system for monitoring a surgical luminaire assembly comprising at least two surgical luminaires, with a monitoring unit, wherein the monitoring unit determines a total power parameter of the exposure of the at least two surgical luminaires into the surgical field.

The second aspect of the present invention takes into account that in most surgical situations, multiple surgical luminaire fields are superimposed or directed simultaneously onto one field, resulting in even higher irradiances and higher temperature loads that can lead to undesirable side effects. Also, the associated maximum illuminance can be so extremely high that it causes glare for the user and quickly tires the eyes.

By determining the total power parameter of the exposure of the at least two surgical luminaires into the surgical field, an excessively large total power parameter of the at least two surgical luminaires in the surgical field can now be detected and the associated problems avoided.

In one possible embodiment of the present invention, the surgical luminaires each have an illuminance of at least 40 kLux and/or a light field diameter of at least 100 mm. Preferably, the surgical luminaires each have an illuminance of at least 80 kLux and/or a maximum light field diameter of at least 200 mm, in particular a maximum illuminance of at least 120 kLux and/or a maximum light field diameter of at least 300 mm.

If the illuminance and/or the light field diameter of the surgical luminaire is adjustable, these indications refer to the fact that the illuminance and/or the light field diameter can be set at least to these values, as already described above for the individual surgical luminaires according to the first aspect.

Furthermore, the absolute and relative range adjustability indications provided above for the first aspect also apply to the surgical luminaires used in the second aspect.

The monitoring unit can be configured here to determine, monitor, display and/or evaluate the total power parameter in the same way as described above for the energy input.

In particular, the monitoring unit can display the total power parameter and/or can monitor for exceedance of a limit value.

In particular, in a possible embodiment of the present invention, it can be provided that the monitoring unit outputs an acoustic and/or optical warning and/or automatically limits or reduces the illuminance when a limit value is exceeded, wherein the limit value is preferably a limit value for the total power parameter and/or a value determined therefrom, and/or wherein the limit value can preferably be adjusted by an operator.

In one possible embodiment of the present invention, the total power parameter may be the total energy input of the at least two surgical luminaires into the surgical field.

In particular, the monitoring unit used according to the second aspect may be designed and may operate as described in more detail above with respect to the first aspect. In particular, the energy input according to the first aspect may be the total energy input of the at least two surgical luminaires into the surgical field.

In particular, the monitoring unit can determine the total energy input as a function of the light field diameter and the illuminance of each of the at least two surgical luminaires.

In one possible embodiment of the present invention, the surgical luminaires each comprise a plurality of light sources, in particular LEDs, wherein the monitoring unit determines the light field diameter and/or the illuminance in each case on the basis of the control of the light sources.

However, other total power parameters are also conceivable, for example the total irradiance, i.e. the joint radiant power of the at least two surgical luminaires per unit area. Preferably, these are also determined on the basis of the control of the light sources.

In one possible embodiment of the present invention, the monitoring unit determines the total power parameter at least in one operating mode under the assumption that the light fields overlap completely. Therefore, a “worst case” is assumed in the context of the monitoring.

In particular, in this case, the acoustic and/or optical warning issued when a limit value is exceeded can serve to draw the surgeon’s attention to the fact that, with the present setting of the two surgical luminaires, the light fields must not overlap or must not overlap completely.

The determination of the total power parameter under the assumption that the light fields overlap completely has the advantage here that no knowledge about the position and/or orientation of the surgical luminaires is required and the determination can also be carried out in particular exclusively on the basis of data which are already available in the control system of the surgical luminaire as control data for the surgical luminaires.

In one possible embodiment of the present invention, the monitoring unit determines the total power parameter, at least in one operating mode, on the basis of information regarding the actual orientation and/or position of the at least two surgical luminaires and/or the actual overlap of the light fields of the at least two surgical luminaires. This avoids unnecessary warnings and/or control interventions when in reality the light fields do not overlap at all.

In one possible embodiment of the present invention, the system comprises an input unit via which an operator can enter the information.

Alternatively or additionally, the system comprises a detection unit by which the information is automatically determined. In particular, one or more sensors can be provided by which the orientation and/or position of the at least two surgical luminaires and/or the actual overlap of the light fields of the at least two surgical luminaires is detected.

In one possible embodiment of the present invention, the monitoring unit determines the energy input and/or the total power parameter at least in one operating mode under the assumption of a predetermined distance of the at least one surgical luminaire from the surgical field.

In one possible embodiment of the present invention, the predetermined distance between a minimum distance L1 and a maximum distance L2 is selected from the parameter specification of the surgical luminaire.

In one possible embodiment of the present invention, the monitoring unit determines the energy input and/or the total power parameter under the assumption of a distance between the surgical luminaire or luminaires and a surgical field in the range between (L1) 600 mm and (L2) 1500 mm.

In one possible embodiment of the present invention, the monitoring unit determines the total power parameter at least in one operating mode on the basis of information regarding an actual distance of the at least one surgical luminaire from the surgical field.

In one possible embodiment of the present invention, the system comprises an input unit via which an operator can enter the information. Alternatively or additionally, the system comprises a detection unit through which the information is automatically determined. In particular, one or more sensors can be provided, by means of which the actual distance of the at least one surgical luminaire from the surgical field is detected.

In one possible embodiment of the present invention, the at least one surgical luminaire comprises a luminaire body and a handle which is arranged on the luminaire body and via which the luminaire body can be oriented.

In one possible embodiment of the present invention, the handle is arranged within a light-emitting region of the luminaire body. The light-emitting region may comprise a plurality of light sources, in particular LEDs, wherein the light sources are preferably arranged around the handle.

In one possible embodiment of the present invention, a main axis of the handle coincides with a main optical axis of the surgical luminaire.

In one possible embodiment of the present invention, the surgical luminaire comprises a plurality of LEDs, the light fields of which at least partially overlap, wherein preferably the size of the light field is adjustable by control of the LEDs.

If two or more surgical luminaires are provided, they are preferably each formed as just described.

In one possible embodiment of the present invention, the surgical luminaire assembly comprises at least two surgical luminaires, each comprising a stand-alone monitoring unit and an interface for communicating with each other, wherein the total power parameter is determined by the monitoring units on the basis of the information transmitted via the interface.

In one possible embodiment of the present invention, the surgical luminaire assembly comprises at least two surgical luminaires, wherein a common monitoring unit is provided, wherein the monitoring unit is preferably integrated into a common control apparatus of the surgical luminaire assembly.

In one possible embodiment of the present invention, the surgical luminaire assembly comprises at least two surgical luminaires, wherein at least one parameter of the at least two surgical luminaires is synchronously adjustable, in particular the light field diameter and/or the illuminance and/or the color setting and/or the switching on and off.

The present invention further comprises a surgical luminaire assembly comprising at least one surgical luminaire and preferably at least two surgical luminaires and a system for monitoring the surgical luminaire assembly as described above.

In one possible embodiment of the present invention, the surgical luminaire assembly comprises a support system via which the at least one and preferably the at least two surgical luminaires can be arranged above an operating table so as to be adjustable in their position and orientation.

In one possible embodiment of the present invention, the monitoring unit comprises a microcontroller and software stored on a non-volatile memory which runs on the microcontroller to implement the functions described above.

For this purpose, the monitoring unit is preferably in communication with a control apparatus of the surgical luminaire or luminaires and receives control data from said control unit. Furthermore, the monitoring unit can be in communication with input and/or output elements.

Furthermore, in one possible embodiment of the present invention, the system comprises a control apparatus comprising a microcontroller and software which is stored on a non-volatile memory and which runs on the microcontroller to implement the control functions described above.

The monitoring unit can be integrated into the control apparatus or formed separately therefrom.

The present invention will now be explained in greater detail on the basis of a drawing and exemplary embodiments.

In the drawing:

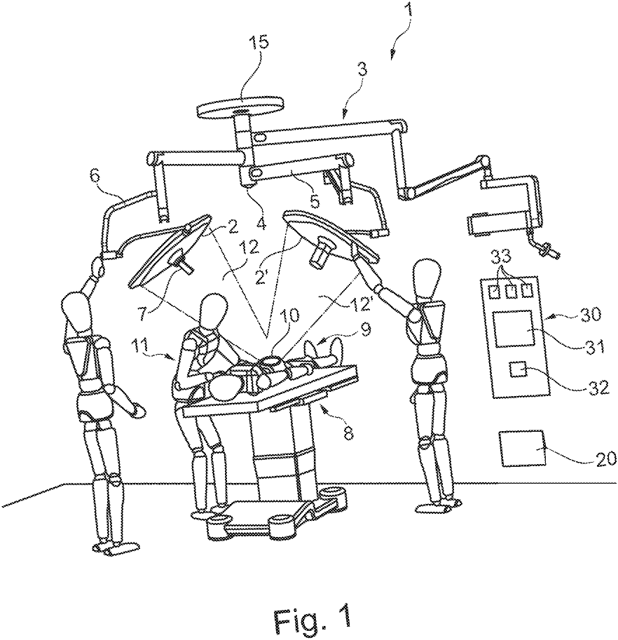

FIG. 1 shows an exemplary embodiment of a surgical luminaire assembly according to the invention with a monitoring unit according to the invention.

FIG. 1 shows an exemplary embodiment of a surgical luminaire assembly 1 according to the invention with a first surgical luminaire 2 and a second surgical luminaire 2′. However, within the scope of the present invention, the surgical luminaire assembly 1 could also comprise only one surgical luminaire or more than two surgical luminaires.

In the exemplary embodiment, the surgical luminaires 2 and 2′ are adjustable in their position and orientation via a support system 3 above an operating table 8. The adjustment is usually done by hand. However, adjustment by means of drives of the support system 3 is also conceivable. In the exemplary embodiment, the support system comprises a ceiling mount 15, via which a central shaft 4 is mounted on the ceiling. Support arms 5 are pivotably arranged on the central shaft 4. The surgical luminaires 2 and 2′ are each arranged on different support arms 5 via further support arm elements 6 and joints, and have a handle 7 on which they can be moved. However, other designs of the support system are also conceivable.

The surgical luminaires 2 and 2′ each comprise a luminaire body which can be oriented via the handle 7 arranged on the luminaire body. In the exemplary embodiment, the handle 7 is arranged within a light-emitting region of the luminaire body.

The light-emitting region in each case comprises a plurality of light sources, in particular LEDs. In the exemplary embodiment, the light-emitting region and/or the light sources are each arranged around the handle 7. In particular, the handle 7 is arranged centrally within the light-emitting region.

In the exemplary embodiment, the main axis of the handle coincides in each case with an optical main axis of the surgical luminaire and therefore points to the region illuminated by the surgical luminaire.

In the exemplary embodiment, the surgical luminaires 2 and 2′ each comprise a plurality of LEDs, the light fields of which at least partially overlap, wherein preferably the size of the light field can be adjusted in each case by control of the LEDs.

The surgical luminaires 2 and 2′ each generate a light field 12 and 12′ respectively. By arranging and orienting the surgical luminaires 2 and 2′ accordingly, the light fields 12 and 12′ can be directed onto a surgical field 10 of the patient 9 lying on the operating table 8 so that they overlap.

In other cases, however, the two light fields 12 and 12′ of the surgical luminaires 2 and 2′ can also be directed towards different regions. For example, in the context of a transplantation, one surgical luminaire can be directed towards the surgical field 10 of the patient 9 lying on the operating table 8, and another surgical luminaire can be directed towards the transplant.

In particular, the surgical luminaires may have one or more of the following operating parameters:
illuminance min. 40 kLux - max. 160 kLux
color temperature adjustable between 3000 - 6000 K
light field size adjustable between 140 - 350 mm diameter
color rendering index > Ra96 (the standard requires > Ra85)
total irradiance at maximum luminosity approx. 540 W/m²
working distance between luminaire and surgical field between 600 mm -1500 mm

FIG. 1 shows a control apparatus 30 via which functions of the surgical luminaires 2 and 2′ can be controlled, in particular brightness adjustment and/or light field size and/or color temperature and/or switching on and off. In the example shown, this control apparatus is mounted on a wall. Alternatively, the control apparatus 30 could also be embodied as a table or mobile version. The control apparatus preferably has input elements 33, for example in the form of switches, actuators and/or a touchscreen. Furthermore, the control apparatus 30 preferably comprises a display 31 on which operating states and/or current setting parameters of the individual surgical luminaires 2 and 2′ can be displayed.

The surgical luminaires 2 and 2′ can be networked with each other and/or with a common controller and/or operating unit by cable and/or wirelessly. Via this communication, it is preferably possible to synchronize functions of the surgical luminaires 2 and 2′, such as brightness adjustment, focus adjustment or color temperature, as well as simultaneous switching on and off.

According to the invention, the surgical luminaire assembly comprises a monitoring unit 20, which is only shown symbolically here. This can be part of a controller of the surgical luminaires, integrated therein and/or external thereto, and/or of the control apparatus 30.

In a first aspect of the present invention, the monitoring unit determines an energy input of at least one surgical luminaire 2 and 2′ into the surgical field 10. The energy input depends on the light field diameter of the particular light field 12 and 12′ and its illuminance. The total energy of each surgical luminaire can be determined by the monitoring unit 20 via these 2 parameters.

This is explained in more detail on the basis of the following example:

A small surgical luminaire with a small light field with a diameter of 160 mm and an illuminance of 160 kLux and a large surgical luminaire with a large light field with a diameter of 350 mm and an illuminance of 160 kLux both have exactly the same irradiance of approx. 550 W/m². However, the total energy input into the surgical field is approx. 3 times higher with the large surgical luminaire than with the small surgical luminaire.

The surgical luminaires preferably each comprise a plurality of light sources, in particular LEDs, wherein the monitoring unit determines the light field diameter and/or the illuminance on the basis of the control of the light sources. A measurement of the light field diameter and/or the illuminance can therefore be omitted. In particular, the light field diameter and/or the illuminance is determined exclusively on the basis of the control of the light sources.

In one possible embodiment and/or in one possible mode of operation of the present invention, the determination of the energy input is carried out entirely on the basis of actuation data alone, without taking measured values into account.

Since the networking of the surgical luminaires means that the monitoring unit 20 knows at all times which parameters are set at each surgical luminaire 2 and 2′, it can either emit a warning signal acoustically or display it visually in the event of critical settings of one or more surgical luminaires. For example, a corresponding display 32 can be provided on the control apparatus 30, for example in the form of a flashing light.

Similarly, the monitoring unit 20 can be configured to automatically reduce or limit the illumination level. Especially if the illuminance of all surgical luminaires is synchronized, the risk of excessive energy/temperature/tissue drying can be significantly reduced.

In one possible embodiment of the invention, this functionality can be switched on or off.

In one possible embodiment of the invention, it can be selected whether a warning sound or display is implemented or both.

In one possible embodiment of the invention, a maximum value for the energy input can be preset by an operator, for example by corresponding input elements of the control apparatus 30.

In one possible embodiment of the invention, the surgical luminaires have an illuminance of at least 40 kLux and/or a light field diameter of at least 140 mm.

In one possible embodiment of the invention, the temperature rise in the region of the surgical field 10 caused by the surgical luminaires 2 and 2′ can be indicated to the operator 11, or he can preset a limit value for the temperature value.

If several surgical luminaires 2 and 2′ are provided, as in the exemplary embodiment, their light fields 12 and 12′ can be superimposed or directed simultaneously onto the surgical field 10. This results in very high irradiances and high temperature loads, which can lead to undesirable side effects. The associated maximum illuminance can also be so extremely high that it causes glare for the user and quickly tires the eyes.

According to the second aspect of the present invention, the monitoring unit therefore determines a total power parameter of the exposure of the at least two surgical luminaires 2 and 2′ into the surgical field 10.

The total power parameter can now be monitored and/or displayed in the same way as described above for the energy input.

The total power parameter may be the jointly achieved illuminance and/or irradiance. Preferably, however, the total energy input of the at least two surgical luminaires 2 and 2′ into the surgical field 10 is also determined here as described according to the first aspect. In particular, this is done via the light field diameter of the particular light field 12 and 12′ and its illuminance.

Using these parameters, a possible maximum system energy can be determined even without information regarding the orientation of the surgical luminaires. This procedure is based on the worst-case scenario that all light fields are directed at one point from a given distance to the surgical luminaire. Here, for example, a distance of one meter can be assumed.

Alternatively or additionally, however, the determination of the total power parameter can also be carried out on the basis of information regarding the actual arrangement of the surgical luminaires 2 and 2′. In particular, it can be provided that an operator of the monitoring unit can specify a certain overlap of light fields and/or can adjust a distance, on the basis of which the determination is then made. This makes individual adjustment and assessment possible.

Alternatively or additionally, the monitoring unit can comprise one or more sensors via which the information regarding the actual arrangement of the surgical luminaires 2 and 2′ is determined, in particular information regarding the overlapping of light fields and/or regarding the distance of the particular surgical luminaires 2 and 2′ from the surgical field 10.

1. A system for monitoring a surgical luminaire assembly, comprising at least one surgical luminaire, with a monitoring unit wherein
the monitoring unit determines an energy input of the at least one surgical luminaire into the surgical field.

2. The system according to claim 1, wherein the monitoring unit determines the energy input as a function of a light field diameter and an illuminance of the at least one surgical luminaire wherein the monitoring unit determines the light field diameter and/or the illuminance on the basis of the actuation of the light sources.

3. The system according to claim 2, wherein the light field diameter of the at least one surgical luminaire is variable by at least 25 mm, and/or wherein the light field diameter of the at least one surgical luminaire is adjustable at least in a light field diameter range between 200 mm and 300 mm, and/or wherein the illuminance of the at least one surgical luminaire is variable by at least 20 kLux, and/or wherein the illuminance of the at least one surgical luminaire is adjustable at least in an illuminance range between 80 kLux and 120 kLux.

4. The system according to claim 2, wherein the monitoring unit displays the energy input into the surgical field and/or a value determined on the basis of the energy input into the surgical field, and/or monitors for exceedance of a limit value.

5. The system according to claim 1, wherein the monitoring unit emits an acoustic and/or optical warning and/or automatically limits or reduces the illuminance when a limit value is exceeded.

6. The system according to claim 4, wherein a monitoring and/or control function of the monitoring unit can be switched on and off, and/or wherein the type of display and/or response to the limit value being exceeded is selectable.

7. The system, according to claim 1, wherein the surgical luminaire assembly comprises at least two surgical luminaires, wherein
the monitoring unit determines a total power parameter of an exposure of the at least two surgical luminaires into the surgical field.

8. The system according to claim 7, wherein the total power parameter is the total energy input of the at least two surgical luminaires into the surgical field, wherein the monitoring unit determines the energy input as a function of a light field diameter and an illuminance of each of the at least two surgical luminaires, wherein the surgical luminaires each comprise a plurality of light sources, wherein the monitoring unit determines the light field diameter and/or the illuminance in each case on the basis of the actuation of the light sources.

9. The system according to claim 8, wherein the monitoring unit determines the total power parameter at least in one operating mode under the assumption that the light fields overlap completely, and/or wherein the monitoring unit determines the total power parameter at least in one operating mode on the basis of information regarding the actual orientation and/or position of the at least two surgical luminaires and/or regarding the actual overlap of the light fields of the at least two surgical luminaires, wherein the system comprises an input unit via which an operator can enter the information and/or a detection unit by which the information is automatically determined.

10. The system according to claim 1, wherein the monitoring unit determines the energy input and/or the total power parameter at least in one operating mode under the assumption of a predetermined distance of the at least one surgical luminaire from the surgical field.

11. The system according to claim 1, wherein the at least one surgical luminaire comprises a luminaire body and a handle which is arranged on the luminaire body, via which the luminaire body can be oriented.

12. System according to claim 1, wherein the surgical luminaire assembly comprises at least two surgical luminaires, each comprising a stand-alone monitoring unit and an interface for communicating with each other, wherein the total power parameter is determined by the monitoring units on the basis of the information transmitted via the interface, or wherein a common monitoring unit is provided.

13. The system according to claim 1, wherein the surgical luminaire assembly comprises at least two surgical luminaires, wherein at least one parameter of the at least two surgical luminaires is synchronously adjustable, in particular the light field diameter and/or the illuminance and/or the color setting and/or the switching on and off.

14. A surgical luminaire assembly, comprising at least one or more surgical luminaires and a system according to claim 1.

15. The surgical luminaire assembly according to claim 14, wherein the surgical luminaire assembly comprises a support system via which the one or more luminaires can be arranged above an operating table so as to be adjustable in their position and orientation.

16. The system according to claim 1 wherein the surgical luminaire has an illuminance of at least 40 kLux and/or a light field diameter of at least 100 mm.

17. The system according to claim 2 wherein the surgical luminaire comprises a plurality of light sources.

18. The system according to claim 4 wherein the value is determined on the basis of a temperature rise of the surgical field.

19. The system according to claim 10 wherein a distance is in the range between 600 mm and 1500 mm is assumed, and/or wherein the monitoring unit determines the total power parameter at least in one operating mode on the basis of information regarding an actual distance of the at least one surgical luminaire from the surgical field, wherein the system comprises an input unit via which an operator can enter the information and/or a detection unit by which the information is automatically determined.

20. The system according to claim 11 wherein the handle is arranged within a light-emitting region of the luminaire body and/or a main axis of the handle coincides with an optical main axis of the surgical luminaire, and/or wherein the surgical luminaire comprises a plurality of LEDs, the light fields of which at least partially overlap, wherein the size of the light field is adjustable by actuation of the LEDs.
